(19) United States
(12) Patent Application Publication
(10) Pub. No.: US 2014/0093842 A1
(43) Pub. Date: Apr. 3, 2014

(54) DENTAL IMPLANT WITH LASER-ETCHED PLATFORM SWITCHING BEVELED COLLAR SURFACE

(71) Applicant: Evollution IP Holdings, Inc., Dover, DE (US)

(72) Inventors: J. Todd STRONG, Vestavia, AL (US); Fred J. MOLZ, Birmingham, AL (US); R. Steven BOGGAN, Birmingham, AL (US); Boyd A. PETERS, Birmingham, AL (US)

(73) Assignee: Evollution IP Holdings, Inc., Dover, DE (US)

(21) Appl. No.: 14/037,150

(22) Filed: Sep. 25, 2013

Related U.S. Application Data
(60) Provisional application No. 61/707,347, filed on Sep. 28, 2012.

(51) Int. Cl. A61C 8/00; A61K 6/04

(52) U.S. Cl. CPC A61C 8/0013; A61C 8/0069; A61K 6/04; A61C 8/0022
USPC 433/174; 433/201.1

(57) ABSTRACT

A dental implant system including a dental implant with a micro-pattern surface treatment and an abutment with a micro-pattern surface treatment. The abutment cooperatively engages the dental implant during use.

### CROSS-REFERENCE TO RELATED APPLICATION

This application claims the benefit of U.S. Provisional Patent Application Ser. No. 61/707,347 filed Sep. 28, 2012, the entirety of which is hereby incorporated by reference herein.

### TECHNICAL FIELD

The present invention relates generally to the field of dental implants, and more particularly to a dental implant having a beveled platform switching collar treated with a micro-pattern surface treatment for improved bone and soft tissue attachment and increased soft tissue volume.

### BACKGROUND

Dental implants are used in the replacement of natural teeth with prosthetic teeth. Dental implants are fitted with abutments to which a crown or other dental restoration is mounted. Traditionally, the diameter of the abutment was preferably selected to generally match the diameter of the implant, which was known as platform matching.

More recently, it has been discovered that “platform switching,” wherein a smaller diameter abutment is mounted to a larger diameter implant, may result in improved bone and soft-tissue response. U.S. Patent Pub. No. 2010/0196852 A1 and U.S. Patent Pub. No. 2012/0077151 A1 are incorporated herein by reference, and provide further details regarding platform switching.

An alternative means of improving tissue response to implant placement provides for the application of ordered repeating microgeometric patterns to bone and tissue interface zones of a dental implant, for example by laser etching alternating ridges and grooves on an implant surface. U.S. Pat. No. 6,419,491 is incorporated herein by reference.

To applicant's knowledge, those of ordinary skill in the art have considered platform switching and micro-pattern etching to be alternative processes, with no known motivation to combine the different techniques. Additionally, platform switching implants typically include a beveled collar having obliquely angled surfaces, which may not be well suited to known laser-etching processes utilized for applying micro-pattern surface treatments to right cylindrical surface zones.

Accordingly, it can be seen that needs exist for improved implants for securing abutments and prosthetic teeth. It is to the provision of improved systems and methods meeting these and other needs that the present invention is primarily directed.

### SUMMARY

The present disclosure provides an improved dental implant system including a dental implant with a micro-pattern surface treatment and an abutment with a micro-pattern surface treatment. The abutment cooperatively engages the dental implant during use.

In certain embodiments, the dental implant micro-pattern surface treatment includes an ordered micro-geometric repetitive surface pattern. The ordered micro-geometric repetitive surface pattern can include alternating ridges and grooves and/or microthreads. Optionally, the alternating ridges and grooves each have a width and a depth on the order of about 2 to about 25 microns.

In other embodiments, the dental implant is made of a material selected from titanium, stainless steel, plastics, ceramics, and other biocompatible metals. In another embodiment, the dental implant also includes a threaded anchor section and a platform switching collar section, wherein the platform switching collar section tapers from a larger dimension to a smaller dimension, and wherein at least a portion of the platform switching collar section comprises the micro-pattern surface treatment. The platform switching collar section can include a bevel of about 30°-60°. Optionally, the platform switching collar section comprises a bevel of about 45°.

In an alternative embodiment, the present disclosure provides a dental implant for complementary engagement with an abutment including a micro-pattern surface treatment. The dental implant includes a micro-pattern surface treatment that aligns with the abutment micro-pattern surface treatment during use.

In certain embodiments, the dental implant tapered collar includes a micro-pattern surface treatment. The abutment tapered collar can include a micro-pattern surface treatment. Optionally, the dental implant micro-pattern surface treatment and the abutment tapered collar micro-pattern surface treatment include an ordered micro-geometric repetitive surface pattern. The ordered micro-geometric repetitive surface pattern can include alternating ridges and grooves and/or microthreads. Optionally, the alternating ridges and grooves each have a width and a depth on the order of about 2 to about 25 microns.

In another embodiment, the abutment includes at least one of the group comprising titanium and alloys thereof, stainless steel, ceramics, biocompatible glass and combinations thereof. In another embodiment, the dental implant includes at least one of the group comprising titanium and alloys thereof, stainless steel, ceramics, biocompatible glass and combinations thereof.

In still a further alternative embodiment, the present disclosure provides a dental implant system including a dental implant and an abutment. The dental implant includes a threaded anchor and a tapered collar tapering from a wide distal section to a narrow proximal section. The abutment includes a base and a tapered collar tapering from a wide distal section to a narrow proximal section. The abutment tapered collar narrow proximal section cooperatively engages the dental implant tapered collar narrow proximal section during use.

In some embodiments, the dental implant includes a tapered collar tapering from a wide distal section to a narrow proximal section, and the abutment comprises a base and a tapered collar tapering from a wide distal section to a narrow proximal section, the abutment tapered collar narrow proximal section cooperatively engages the dental implant tapered collar narrow proximal section during use.

In other embodiments, the dental implant micro-pattern surface treatment and the abutment micro-pattern surface treatment include an ordered micro-geometric repetitive surface pattern. Optionally, the ordered micro-geometric repetitive surface pattern comprises alternating ridges and grooves and/or microthreads.

The disclosure also provides a method of causing growth of gum tissue around a dental implant in a subject including implanting a dental implant for complementary engagement with an abutment in the mouth of the subject comprising a micro-pattern surface treatment, the dental implant comprising a micro-pattern surface treatment that aligns with the abutment micro-pattern surface treatment during use, wherein the dental implant comprises a threaded anchor section and a platform switching collar section, wherein the platform switching collar section tapers from a larger dimension to a smaller dimension, and wherein at least a portion of the platform switching collar section comprises the micro-pattern surface treatment.

In certain embodiments, the dental implant micro-pattern surface treatment includes an ordered micro-geometric repetitive surface pattern. The ordered micro-geometric repetitive surface pattern can include alternating ridges and grooves and/or microthreads. Optionally, the alternating ridges and grooves each have a width and a depth on the order of about 2 to about 25 microns.

In other embodiments, the dental implant is made of a material selected from titanium, stainless steel, plastics, ceramics, and other biocompatible metals. In another embodiment, the dental implant also includes a threaded anchor section and a platform switching collar section, wherein the platform switching collar section tapers from a larger dimension to a smaller dimension, and wherein at least a portion of the platform switching collar section comprises the micro-pattern surface treatment. The platform switching collar section can include a bevel of about 30°-60°. Optionally, the platform switching collar section comprises a bevel of about 45°.

In certain embodiments, the gum tissue covers the implant so that the implant is not visible in the mouth of the subject. In other embodiments, the gum tissue covers at least a portion of the abutment so that when a prosthetic tooth is installed on the abutment the abutment is not visible in the mouth of the subject.

### BRIEF DESCRIPTION OF THE FIGURES

FIG. 1 shows three dental implants according to an example embodiment, installed within gum tissue and mounting abutments.

FIG. 2 shows an enlarged side view of one of the dental implants and abutments shown in FIG. 1.

FIG. 3 shows an enlarged front view of one of the dental implants and abutments shown in FIG. 1.

FIG. 4 shows an isolated top perspective view of one of the dental implants shown in FIG. 1.

FIG. 5 shows a side view of the dental implant shown in FIG. 4.

FIG. 6 shows a top view of the dental implant shown in FIG. 4.

FIG. 7 shows a bottom view of the dental implant shown in FIG. 4.

FIG. 8 shows an isolated top perspective view of the abutments shown in FIG. 1.

FIG. 9 shows a back view of the abutment shown in FIG. 8.

FIG. 10 shows a front view of the abutment shown in FIG. 8.

FIG. 11 shows a side view of the abutment shown in FIG. 8.

FIG. 12 shows a top view of the abutment shown in FIG. 8.

FIG. 13 shows a bottom view of the abutment shown in FIG. 8.

FIG. 14A shows an isolated top perspective view of one embodiment of the dental implant.

FIG. 14B shows a side view of one embodiment of the dental implant.

FIG. 14C shows a side view of one embodiment of the dental implant joined with an abutment.

FIG. 15A shows the dimensions for implant length and ordered micro-geometric repetitive surface pattern zone.

FIG. 15B shows the dimensions for the platform switch.

FIG. 15C shows the dimensions for body diameter and prosthetic connection width.

### DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

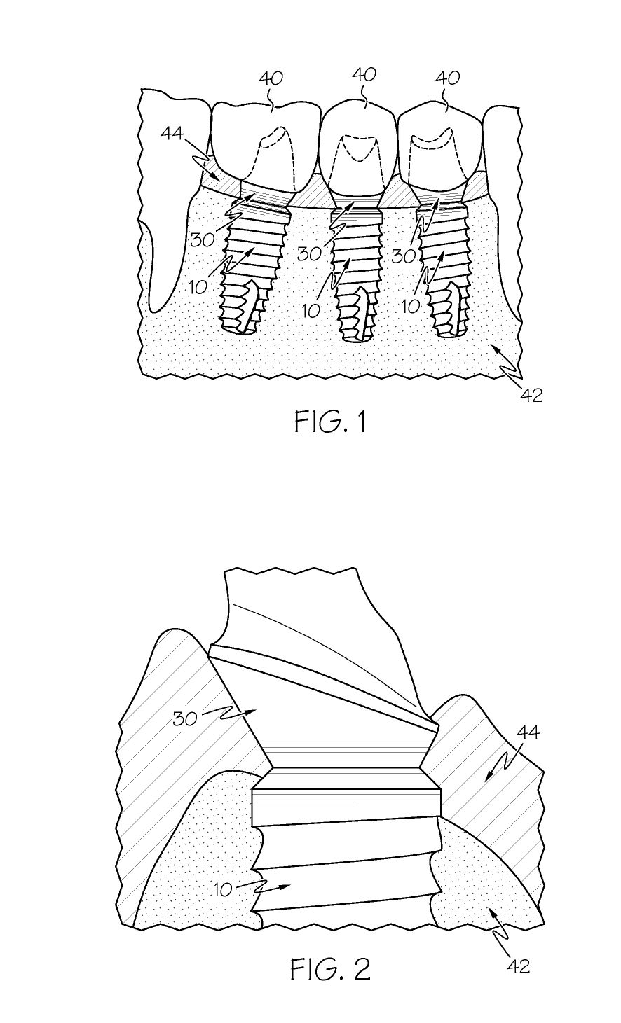

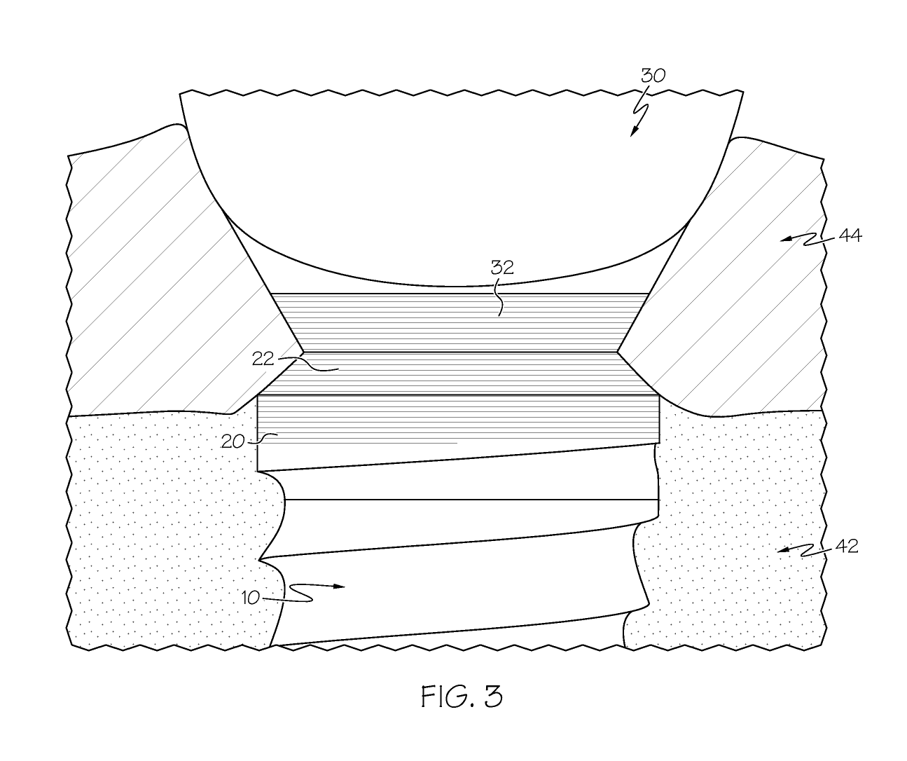

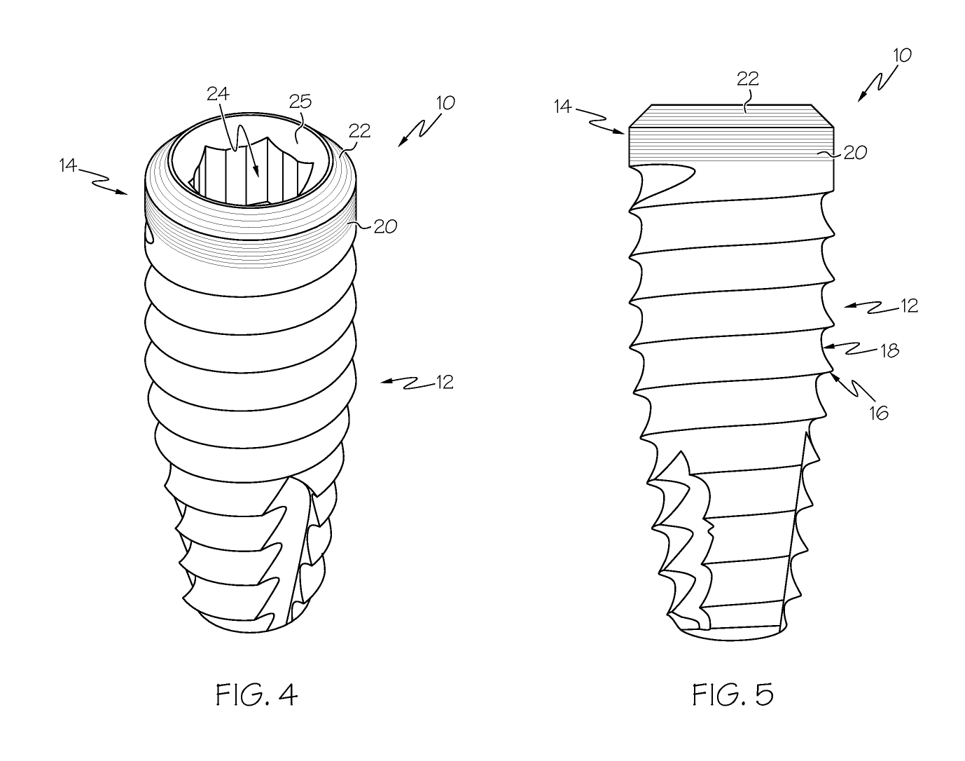

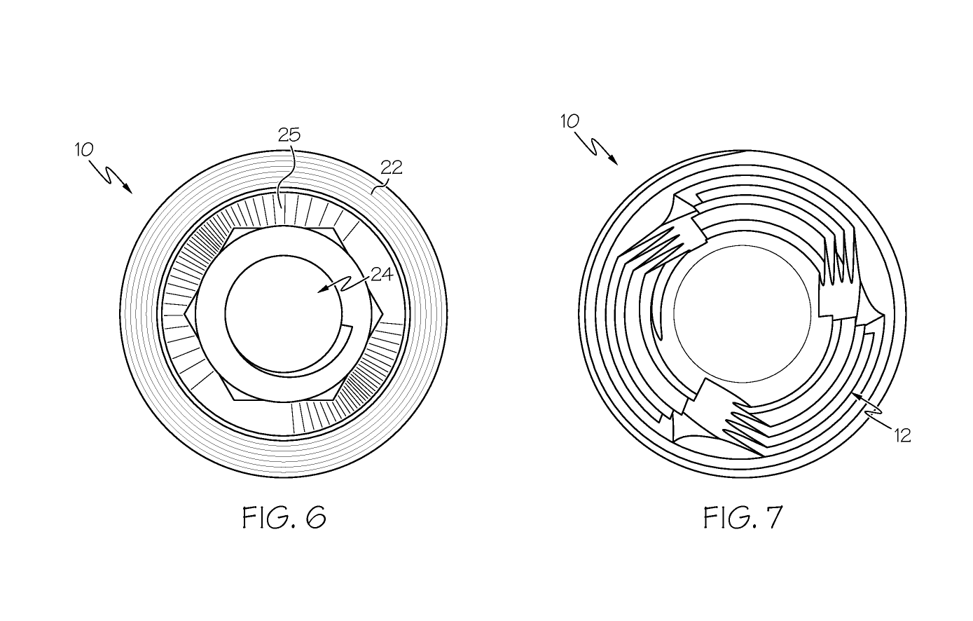

FIGS. 1-7 depict an embodiment of an improved dental implant 10 for supporting an abutment 30 onto which a prosthetic tooth 40 is mounted. The implant 10 has a threaded anchor section 12 and a platform switching collar section 14. At least a portion of the platform switching collar section 14 has a micro-pattern surface treatment applied thereon to the outer surface. When the implant 10 is secured within osteomy crestal bone 42, the bone and soft gum tissue 44 will grow into and grip the microgeometric surface pattern zone on the collar section 14.

The platform switching collar section 14 includes a cylindrical collar 20 and a mouth 22 that tapers from a larger outside diameter at a distal section to a smaller outside diameter at a proximal section thereof. In example embodiments, the cylindrical collar portion 20 has a height about equal to or greater than the height of the mouth portion 22. In embodiments in which the collar 20 has a height greater than the mouth portion 22, the collar 20 has a height between about 1.1 to 2.0 times the height of the mouth portion 22. In other embodiments, the collar 20 has a height between about 1.1 to 1.5 times the height of the mouth portion 22. In other embodiments, the collar 20 has a height of about 1.1, 1.2, 1.3, 1.4, 1.5, 1.6, 1.7, 1.8, 1.9 or 2.0 times the height of the mouth portion 22. The threaded anchor section 12 extends from the collar section 14 and has a plurality of alternating ridges 16 and troughs 18. The threaded anchor section 12 is tapered from a larger outer diameter at a proximal section to a smaller outer diameter at a distal section thereof.

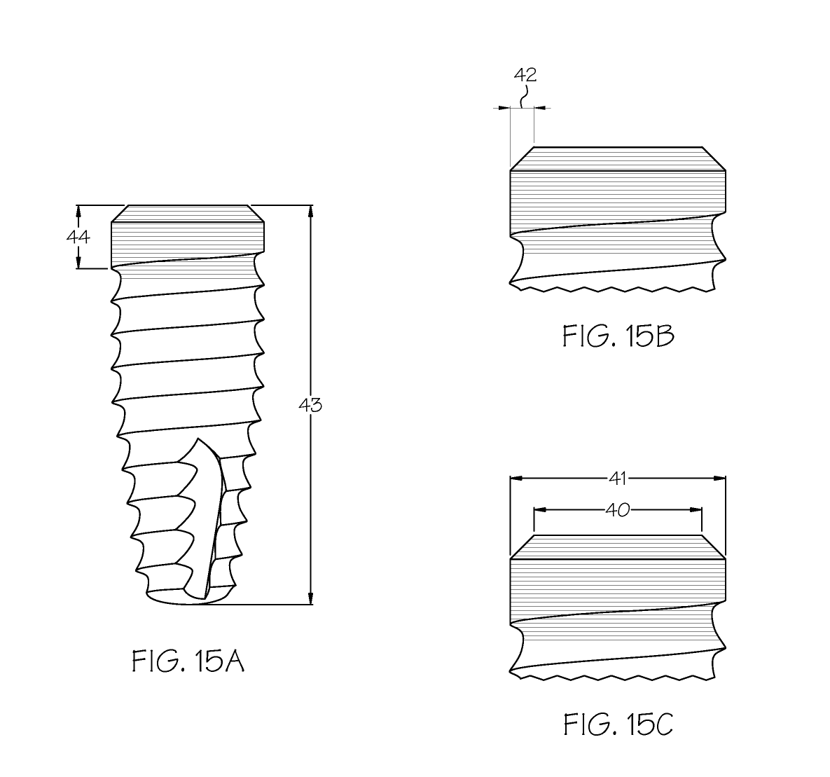

In certain embodiments, the implant has specific dimensions, as shown in FIG. 15. In some embodiments, the implant has a length 43 between about 5 mm and 20 mm. In other embodiments, the implant has a length 43 of between about 7.5 mm and 15 mm, 9 mm and 12 mm, or 10.5 mm and 15 mm. In other embodiments, the implant has a length 43 of about 5.0, 7.5, 9.0, 10.5, 12.0 or 15.0 mm.

In other embodiments, the ordered micro-geometric repetitive surface pattern zone 44 of the implant is located only at the proximal end of the implant. However, according to other embodiments, an ordered micro-geometric repetitive surface pattern can extend to cover most or all of the implant. In embodiments in which the ordered micro-geometric repetitive surface pattern zone 44 is only located at the proximal end of the implant, as shown in FIG. 15A. In certain embodiments, the ordered micro-geometric repetitive surface pattern zone 44 is between about 1.5 and 2.5 mm in height. In other embodiments, the ordered micro-geometric repetitive surface pattern zone 44 is between about 1.8 and 2.1, 1.6 and 2.2, or 1.6 and 2.3 mm in height. In other embodiments, the ordered micro-geometric repetitive surface pattern zone 44 is about 1.8 or 2.1 mm in height.

The implants described herein also provide a platform switch 42, as shown in FIG. 15B. The horizontal distance 42 between the cylindrical collar 20 to the opening of the tapered collar mouth 22 can be between about 0.3 and 0.7 mm. In other embodiments, the horizontal distance 42 between the cylindrical collar 20 to the opening of the tapered collar mouth 22 is between about 0.4 and 0.6 mm. In other embodiments, the horizontal distance 42 between the cylindrical collar 20 to the opening of the tapered collar mouth 22 is about 0.4, 0.5 or 0.6 mm.

In certain embodiments, the body diameter 41, as shown in FIG. 15C, is between about 1.5 and 5.0 mm. In other embodiments, the body diameter 41 is between about 2.0 and 3.9, 2.0 and 2.8, 2.8 and 3.1 or 3.1 and 3.9 mm. In other embodiments, the body diameter 41 is about 2.0, 2.8, 3.1 or 3.9 mm. The prosthetic connection width 40 can be between about 1.5 and 3.0 mm. In other embodiments, the prosthetic connection width 40 is between about 2.0 and 2.7, 2.0 and 2.1 or 2.1 and 2.7 mm. In other embodiments, the prosthetic connection width 40 is about 2.0, 2.1 or 2.7 mm.

In example embodiments, the micro-pattern surface treatment comprises an ordered micro-geometric repetitive surface pattern, for example in the form of alternating ridges and grooves, each having a width and a depth on the order of about 0.1 micron to 100 microns, and more preferably about 2 microns to about 25 microns, and having pattern geometries as shown for example in U.S. Pat. No. 5,816,813, U.S. Pat. No. 6,419,491, and/or U.S. Pat. No. 6,547,564, which are incorporated herein by reference. In alternate embodiments, different pattern geometries and/or pattern element dimensions are provided within the scope of the invention. For example, a microthread surface pattern may be utilized.

The tapered collar mouth 22 optionally has an outer beveled surface of between 0° and 89° from the horizontal plane. The horizontal plane described in these embodiments can be defined by a horizontal line defined by the stop of the implant when viewed from the side, as shown for example in FIG. 15. In other embodiments, the outer beveled surface is arranged at an angle of between about 10°-20°, 20°-30°, 30°-40°, 40°-50°, 60°-70°, 70°-80°, 80°-89°, 30°-60° or 20°-70° above the horizontal plane. In certain embodiments, the outer beveled surface is arranged at an angle of about 0°, 1°, 2°, 3°, 4°, 5°, 10°, 15°, 20°, 25°, 30°, 35°, 40°, 45°, 50°, 55°, 60°, 65°, 70°, 75°, 80°, 85°, 86°, 87°, 88° or 89° above the horizontal plane. In certain embodiments, the outer beveled surface forms a straight line between the top of the cylindrical collar 20 to the top of the tapered collar mouth 22. In other embodiments, the outer beveled surface forms an arc between the top of the cylindrical collar 20 to the top of the tapered collar mouth 22. This arc can curve out from the implant or curve into the implant.

In other embodiments, the outer surface of the tapered collar mouth 22 includes one or more steps instead of a straight surface extending from the cylindrical collar 20 to the top of the tapered collar mouth 22. In certain embodiments, these steps form approximate 90° angles from the straight surface of the cylindrical collar 20. So, instead of an outer beveled surface that extends at an angle to the horizontal all the way to the top of the tapered collar mouth 22, the outer surface of the tapered collar mouth can include one or more steps that lead from the straight surface of the cylindrical collar 20 to the top of the tapered collar mouth 22. In other embodiments, the steps have between 90° and 120°, 90° and 110°, 90° and 100°, 100° and 110°, 100° and 120° or 110° and 120° angles between the vertically rising portion of each step and the horizontally running portion of each step. In certain embodiments, the vertically rising portion of each step rises parallel to the straight surface of the cylindrical collar 20. In other embodiments, the vertically rising portion of each step can be discerned by rising more vertically than it runs horizontally. In certain embodiments, the horizontally running portion of each step extends perpendicularly to the straight surface of the cylindrical collar 20. In other embodiments, the horizontally running portion of each step can be discerned by running more horizontally than it rises vertically. In certain embodiments, the outer beveled surface 22 includes 1, 2, 3, 4, 5, 6, 7, 8, 8, 10 or more steps.

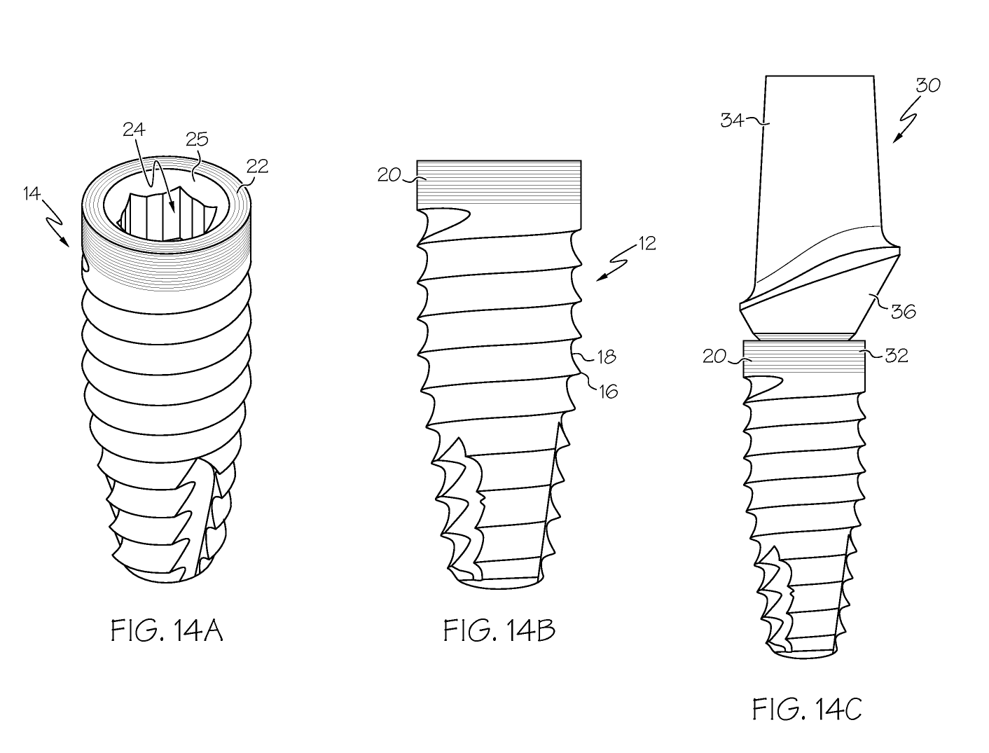

In another embodiment, shown for example in FIG. 14, the tapered collar mouth 22, is either parallel with the horizontal plane of the implant or is beveled so that it decreases in height from the outer rim of the implant to the upper end of the inner beveled surface 25. As shown in FIG. 14B the upper edge of the implant forms a horizontal plane of the implant. In this embodiment, the cylindrical collar 20 has an ordered micro-geometric repetitive surface pattern leading up to the top of the implant. Then, the tapered collar mouth 22, is either flush with this horizontal plane or angled downward toward the lower inner beveled surface 25, which can be at the same height in the implant as the upper edge of the tapered collar mouth 22, or below it. In some embodiments, the tapered collar mouth 22 also has an ordered micro-geometric repetitive surface pattern. In certain embodiments, from where it meets the cylindrical collar the tapered collar mouth 22 tapers downwardly at an angle between about 1° and 89° below the horizontal plane. In other embodiments, this angle is between 10°-20°, 20°-30°, 30°-40°, 40°-50°, 60°-70°, 70°-80°, 80°-89°, 30°-60° or 20°-70° below the horizontal plane. In certain embodiments, the outer beveled surface is arranged at an angle of about 0°, 1°, 2°, 3°, 4°, 5°, 10°, 15°, 20°, 25°, 30°, 35°, 40°, 45°, 50°, 55°, 60°, 65°, 70°, 75°, 80°, 85°, 86°, 87°, 88° or 89° below the horizontal plane. In certain embodiments, the outer beveled surface forms a straight line between the top of the cylindrical collar 20 to upper end of the inner beveled surface 25. In other embodiments, the outer beveled surface forms an arc between the top of the cylindrical collar 20 to the upper end of the inner beveled surface 25. This arc can curve out from the implant or curve into the implant.

In other embodiments, the outer surface of the tapered collar mouth 22 includes one or more steps instead of a straight surface extending from the top of the cylindrical collar 20 to upper end of the inner beveled surface 25. In certain embodiments, these steps each have approximate 90° angles from the straight surface of the cylindrical collar 20. So, instead of an outer beveled surface that extends at an angle to the horizontal all the way to the upper end of the inner beveled surface 25, the outer surface of the tapered collar mouth includes one or more steps that lead from the straight surface of the cylindrical collar 20 to the abutment receiver 24. In other embodiments, the steps have between 90° and 120°, 90° and 110°, 90° and 100°, 100° and 110°, 100° and 120° or 110° and 120° angles between the vertically rising portion of each step and the horizontally running portion of each step. In certain embodiments, the vertically rising portion of each step rises parallel to the straight surface of the cylindrical collar 20. In other embodiments, the vertically rising portion of each step can be discerned by rising more vertically than it runs horizontally. In certain embodiments, the horizontally running portion of each step extends perpendicularly to the straight surface of the cylindrical collar 20. In other embodiments, the horizontally running portion of each step can be discerned by running more horizontally than it rises vertically. In certain embodiments, the outer beveled surface 22 includes 1, 2, 3, 4, 5, 6, 7, 8, 8, 10 or more steps.

In one specific embodiment, the outer bevel surface 22 is arranged at an angle of about 45° from the horizontal plane, and provides an implant—abutment receiver 24 diameter mismatch of at least about 0.8 mm, providing a width of platform switch of at least about 0.4 mm about the periphery of the abutment when mounted in a centered configuration on the implant. In alternate embodiments, the platform switching collar 14 comprises a radiused transition from a larger outside diameter at a proximal section to a smaller outside diameter at a distal section thereof. In further alternate embodiments, a flat surface (i.e., 0° or 180°) or a stepped surface having at least two segments of differing dimension defines the platform switching collar section.

The implant 10 may comprise titanium, stainless steel, plastics, ceramics, and/or other biocompatible metals, alloys, composites or other materials, as well as combinations thereof, and may optionally further comprise one or more surface coatings. The micro-pattern surface treatment may be applied by laser etching, acid etching, mechanical etching or micro-machining, photolithography, or other surface treatment processes.

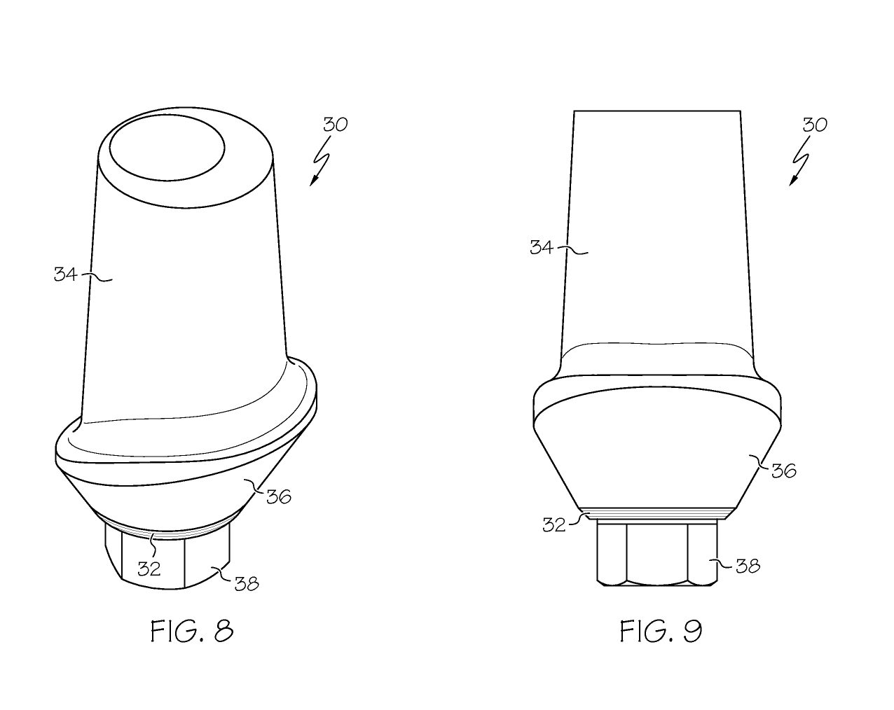

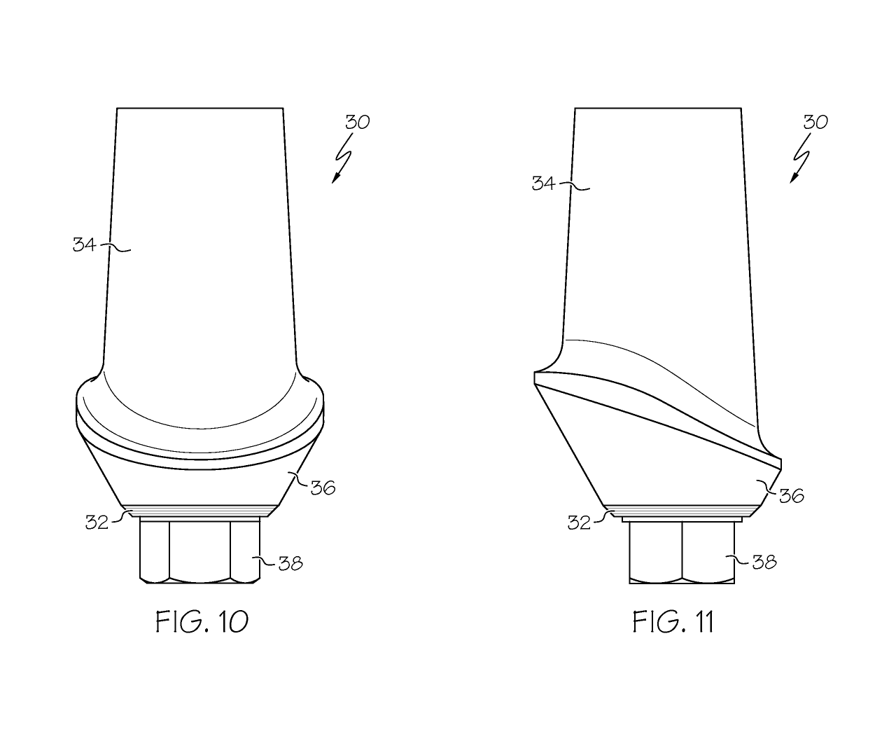

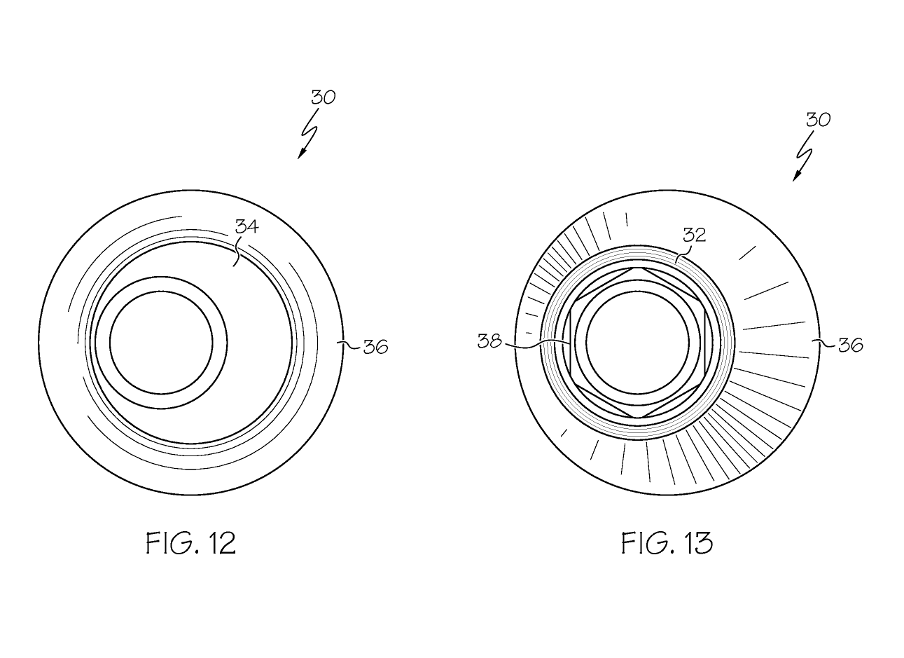

As particularly depicted in FIGS. 1-3 and 9-13, an alternative embodiment also comprehends an implant system comprising the dental implant 10 as described, in combination with an abutment 30 proportioned for complementary engagement with the implant. The abutment 30 can have a neck mount 34 that tapers from a larger outer diameter at a proximal section to a smaller outer diameter at a distal section. The neck mount 34 extends from a base 36 that can have an ergonomic shape configured to be accepted within soft gum tissue 44. The base 36 can be tapered from a larger outer diameter distal end to a smaller outer diameter proximal end. The abutment base 36 has an insert 38 that secures within the implant—abutment receiver 24. The abutment insert 38 has a shape and diameter designed to fit snugly and not rotate within the abutment—insert receiver 24. When the abutment 30 is secured to the implant 10, the combined outer surfaces of the base 36 and the collar mouth 22 form a V-shape. In other embodiments, the combined outer surfaces can be flush, as shown in FIG. 14C.

The base material of the abutment 30 is selected from the group consisting of titanium and alloys thereof, stainless steel, ceramics, biocompatible glass and combinations thereof. The micro-pattern surface treatment may be applied by laser etching, acid etching, mechanical etching or micro-machining, photolithography, or other surface treatment processes.

As particularly shown in FIGS. 1-3, the implant threaded anchor section 12 is secured into the bone 42 below the soft gum tissue 44. The anchor section 12 can be secured by drilling a hole into the bone 42 and rotatably screwing the threaded surface within the hole. Alternatively, the implant anchor section 12 can be inserted within the bone 42 to a depth such that the collar section 20 fits within the bone and the mouth 22 sits above the bone. The abutment 30 is secured to the implant 10, for example by inserting the abutment insert 38 into the implant—abutment receiver 24. When the abutment 30 is secured to the implant 10, the volume of the gum tissue 44 increases over the V-shape between the base 36 and the mouth 22.

The abutment base 36 optionally includes a collar section 32 having an ordered microgeometric surface pattern applied thereon. In example forms, the ordered microgeometric surface pattern comprises alternating ridges and grooves, and/or a microthread geometry, for example each having a width and a depth on the order of about 0.1 micron to 100 microns, and more preferably about 2 microns to about 25 microns. When the abutment 30 is secured to the implant 10, the abutment collar section 32 aligns with the implant collar mouth 22 to provide a consistent microgeometric surface pattern across the implant and the abutment. When the implant 10 and abutment 30 are secured within the bone 42, the bone and gum tissue 44 will grow into and grip the microgeometric surface pattern collars 14, 32.

While the invention has been described with reference to preferred and example embodiments, it will be understood by those skilled in the art that a variety of modifications, additions and deletions are within the scope of the invention, as defined by the following claims.

What is claimed is:

1. A dental implant for complementary engagement with an abutment comprising a micro-pattern surface treatment, the dental implant comprising a micro-pattern surface treatment that aligns with the abutment micro-pattern surface treatment during use.

2. The dental implant of claim 1, wherein the dental implant micro-pattern surface treatment comprises an ordered micro-geometric repetitive surface pattern.

3. The dental implant of claim 2, wherein the ordered micro-geometric repetitive surface pattern comprises alternating ridges and grooves and/or microthreads.

4. The dental implant of claim 3, wherein the alternating ridges and grooves each have a width and a depth on the order of about 2 to about 25 microns.

5. The dental implant of claim 1, wherein the dental implant comprises at least one from the group comprising titanium, stainless steel, plastics, ceramics, and other biocompatible metals.

6. The dental implant of claim 1, further comprising a threaded anchor section and a platform switching collar section, wherein the platform switching collar section tapers from a larger dimension to a smaller dimension, and wherein at least a portion of the platform switching collar section comprises the micro-pattern surface treatment.

7. The dental implant of claim 6, wherein the platform switching collar section comprises a bevel of about 30°-60°,

8. The dental implant of claim 7, wherein the platform switching collar section comprises a bevel of about 45°.

9. A dental implant system comprising:
a dental implant comprising a threaded anchor and a tapered collar tapering from a wide distal section to a narrow proximal section; and
an abutment comprising a base and a tapered collar tapering from a wide distal section to a narrow proximal section, the abutment tapered collar narrow proximal section cooperatively engages the dental implant tapered collar narrow proximal section during use.

10. The dental implant system of claim 9, wherein the dental implant tapered collar comprises a micro-pattern surface treatment.

11. The dental implant system of claim 10, wherein the abutment tapered collar comprises a micro-pattern surface treatment.

12. The dental implant system of claim 11, wherein the dental implant micro-pattern surface treatment and the abutment tapered collar micro-pattern surface treatment comprise an ordered micro-geometric repetitive surface pattern.

13. The dental implant system of claim 12, wherein the ordered micro-geometric repetitive surface pattern comprises alternating ridges and grooves and/or microthreads.

14. The dental implant system of claim 13, wherein the alternating ridges and grooves each have a width and a depth on the order of about 2 to about 25 microns.

15. The dental implant system of claim 9, wherein the abutment comprises at least one of the group comprising titanium and alloys thereof, stainless steel, ceramics, biocompatible glass and combinations thereof.

16. The dental implant system of claim 9, wherein the dental implant comprises at least one from the group comprising titanium, stainless steel, plastics, ceramics, and other biocompatible metals.

17. A dental implant system comprising:
a dental implant comprising a micro-pattern surface treatment; and
an abutment comprising a micro-pattern surface treatment, the abutment cooperatively engages the dental implant during use.

18. The dental implant system of claim 17, wherein the dental implant comprises a tapered collar tapering from a wide distal section to a narrow proximal section, and the abutment comprises a base and a tapered collar tapering from a wide distal section to a narrow proximal section, the abutment tapered collar narrow proximal section cooperatively engages the dental implant tapered collar narrow proximal section during use.

19. The dental implant system of claim 17, wherein the dental implant micro-pattern surface treatment and the abutment micro-pattern surface treatment comprise an ordered micro-geometric repetitive surface pattern.

20. The dental implant system of claim 19, wherein the ordered micro-geometric repetitive surface pattern comprises alternating ridges and grooves and/or microthreads.

21. A method of causing growth of gum tissue around a dental implant in a subject with a mouth, the method comprising:
implanting a dental implant in the mouth of the subject for complementary engagement with an abutment comprising a micro-pattern surface treatment, the dental implant comprising a micro-pattern surface treatment that aligns with the abutment micro-pattern surface treatment during use, wherein the dental implant comprises a threaded anchor section and a platform switching collar section, wherein the platform switching collar section tapers from a larger dimension to a smaller dimension, and wherein at least a portion of the platform switching collar section comprises the micro-pattern surface treatment.

22. The method of claim 21, wherein the dental implant micro-pattern surface treatment comprises an ordered micro-geometric repetitive surface pattern.

23. The method of claim 22, wherein the ordered micro-geometric repetitive surface pattern comprises alternating ridges and grooves and/or microthreads.

24. The method of claim 23, wherein the alternating ridges and grooves each have a width and a depth on the order of about 2 to about 25 microns.

25. The method of claim 21, wherein the dental implant comprises at least one from the group comprising titanium, stainless steel, plastics, ceramics, and other biocompatible metals.

26. The method of claim 21, further comprising a threaded anchor section and a platform switching collar section, wherein the platform switching collar section tapers from a larger dimension to a smaller dimension, and wherein at least a portion of the platform switching collar section comprises the micro-pattern surface treatment.

27. The method of claim 26, wherein the platform switching collar section comprises a bevel of about 30°-60°

28. The method of claim 27, wherein the platform switching collar section comprises a bevel of about 45°.

29. The method of claim 21, wherein the gum tissue covers the implant so that the implant is not visible in the mouth of the subject.

30. The method of claim 29, wherein the gum tissue covers at least a portion of the abutment so that when a prosthetic tooth is installed on the abutment the abutment is not visible in the mouth of the subject.
